(19) United States
(12) Patent Application Publication
(10) Pub. No.: US 2014/0003996 A1
(43) Pub. Date: Jan. 2, 2014

(54) METHOD FOR FILTERING ODORS OUT OF AN AIR FLOW, AND FILTERING DEVICE EQUIPPED WITH AN ODOR FILTER

(71) Applicant: Manfred H. LANGNER, Frankfurt am Main (DE)

(72) Inventor: Manfred H. LANGNER, Frankfurt am Main (DE)

(21) Appl. No.: 14/011,861

(22) Filed: Aug. 28, 2013

Related U.S. Application Data
(63) Continuation of application No. 11/718,102, filed on Jun. 8, 2009.

(30) Foreign Application Priority Data
Oct. 30, 2004 (DE) 10 2004 053 030.0

(51) Int. Cl. A61L 9/22

(52) U.S. Cl. CPC A61L 9/22
USPC 422/5; 422/121

(57) ABSTRACT

A method for filtering odors out of an air flow during which an air now containing odorous substances is guided through a filtering device equipped with an odor filter, whereby the odor filter has activated carbon or a similar substance serving as filtering material. As an additional method step, the inventive method provides that the particles contained in the air flow are at least partially ionized and/or dissociated before entering the odor filter.

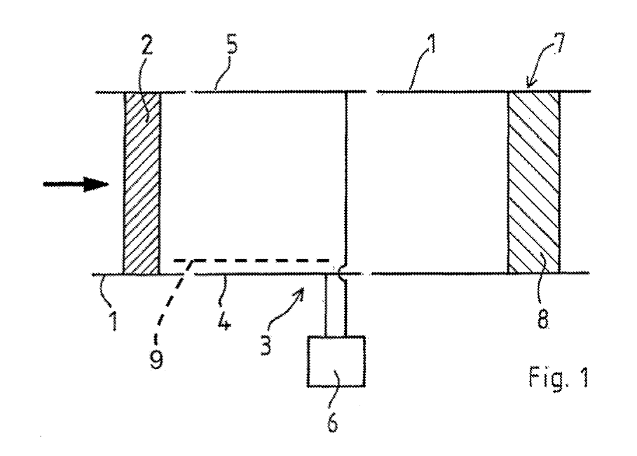

The present application is a continuation of U.S. application Ser.. No. 11/718,102, filed Apr. 27, 2007, which is the U.S. national stage of International Application PCT/EP2005/011526, filed Oct. 27, 2005, which claims priority to DE 10 2004 053 030.0, filed Oct. 30, 2004, all of which are incorporated by reference herein.

The invention relates to a method for filtering odors out of an airflow, during which an airflow containing odorous substances is guided through a filtering device equipped with an odor filter, whereby the odor filter comprises activated carbon or a similar substance serving as filtering material.

Filters equipped with an odor filter are regularly used in ventilation systems, for example, when the air to be carried off contains large amounts of odorous substances and said odorous substances are nor to he released directly, at least not in the present concentration, into the environment. Typical areas of application for odor filters usually are related to heating and preparing foodstuffs. Odor filters are often used in chemical or paint processing industries as well.

Depending on the required filter properties, it is possible to use different filtering processes such as solvent flushing, electrostatic filtering methods, plasma filters or, in most cases, active-carbon containing filtering materials.

For example, ventilation hoods are used in private household kitchens, in which the outgoing air above the stove is suctioned off, is guided through a filtering system and then is guided back into the room in which the stove is located. Apart from mechanical filtering devices that are often used to filter out small particles such as grease droplets or steam drops from the outgoing air, kitchen ventilation hoods advantageously comprise additional odor filters made of active carbon or similar filtering material. Odor filters are to ensure that odorous substances contained in the outgoing air that often are not captured by the filter system for grease droplets and steam are prevented from passing through the vent and being released directly into the environment again.

Most known odor filers are based on filtering material that cannot be regenerated or is difficult to generate, such as active carbon or such, whose filtering effect usually decreases over time, which is why the odor filters mast be replaced or renewed in certain time intervals. While the intervals for replacing odor filters and the associated cots allow for an economic sensible use of kitchen ventilation hoods that are designed for kitchens in private households, ventilation hoods used in commercial or industrial applications often require large amounts of filtering material that must be replaced and renewed in short tune intervals.

The effectiveness of odor filters usually can be increased only by increasing the amount of the filtering material or by increasing the size of the odor filter. Often it is impossible to achieve satisfactory filtering effectiveness for odorous substances in commercially used ventilation hoods, for example in restaurants or commercial kitchens, due to the high share of odorous substances in the outgoing air, without resulting, in an undesirable, unacceptable reduction of the transport volume of the outgoing air. This reduction is due to the flow resistance caused by the amount of the filtering material.

It is desirable to provide a method for filtering odorous substances out of an airflow so that the effectiveness is improved. If possible, the filtering method is not to significantly deteriorate the flow resistance when the air is passed through.

According to an aspect of the present invention, the particles contained in the airflow are at least partially ionized and/or dissociated before entering the odor filter. It was found that the inventive combination of two filtering methods provides for significantly improved filtering effectiveness than the separate use of one or the other filtering method would render obvious.

Either due to direct dissociation or caused by the ionization and subsequent reaction, odorous substances or odor molecules are converted into reaction products that could be odor neutral. Such odor-neutral reaction products do not necessarily have to be filtered out of the airflow in order to avoid undesired odors. In addition, the accumulation and retention of energetically energized reaction products together with odorous substances still contained in the airflow due to the longer dwell time in the subsequent odor filter facilitates another reaction with odorous substances that are converted, at least partially, into odor-neutral reaction products. Only the remaining, considerably reduced amount of originally present odorous substances must be removed from and filtered out of the airflow by the odor filter. This delays an undesirable saturation of the active carbon containing odor filter so that an improved filtering effectiveness is achieved accompanied by reduced maintenance requirements.

In an especially advantageous manner, plasma is generated in the airflow or electric discharges are generated prior to entering the odor filter.

In practical tests, the odorous substance concentration in the outgoing air directly above several deep fryers in a fast food restaurant was compared with the odorous substance concentration of the outgoing air after the outgoing air was subjected to a filtering method according to the invention. While the filtering effect of an active carbon odor filter as well as the filtering, effect of a plasma-based odor filter was able to produce a reduction of the odorous substance concentration by a mean factor of 3 to 5, the combination according to the invention comprised of a filtering method by means of an odor filter with active carbon in conjunction with a preceding ionization or dissociation of at least a part of the particles contained in the outgoing airflow and in particular of the odorous substances, resulted in an odor reduction by a factor of 50 to considerably above 100.

The ionized or dissociated odorous substances can be filtered out of the airflow in a substantially more effective manner with filtering material such as active carbon than would be possible without the additional ionization or dissociation. At the same time, the active carbon is used up in a reduced manner or the effect of an active carbon filter over time is reduced more slowly so that the time intervals in which the filtering material comprised of active carbon must be replaced or renewed are longer despite an improved filtering effectiveness for odors.

It has been known that in particular small particles that cannot or can only be removed insufficiently from the outgoing air with customary mechanical filter devices can easily be separated and removed by generating a plasma. Different methods for filtering based on plasma generation are known and are based on the generation of atmospheric plasma or dielectrically impeded discharge between two plane electrodes.

According to one embodiment of the inventive thought, the airflow is subjected to radiation with high-energetic photons prior to entering the odor filter. The generation of sufficiently energy-rich, for example ultraviolet (UV) light is easily accomplished in a ventilation hood in the area of the airflow. Depending on the wavelength of the generated UV light, different molecules, of particles comprised of a plurality of molecules, are ionized or dissociated and can partially be removed from the outgoing airflow prior to reaching the odor filter with active carbon. The radiation with UV light above all allows the destruction of long chain molecules and odorous substances so that the dissociated residual molecules no longer form odorous substances. It also is possible to generate highly reactive, partially energized residual molecules that react with odorous substances and convert them into odor-neutral reaction products in the process.

Energized, ionized or dissociated particles or molecules are captured in the active carbon filtering material as well so that another reaction of odor molecules or odorous substances that have not oxidized up until now occurs, further improving the filtering effect. Additionally, ozone often is produced in the above process for the ionization or dissociation of the particles contained in the airflow. Within a short period of time, the ozone reacts with other particles contained in the airflow and an oxidation reaction can occur. The ozone is retained in the active carbon filtering material together with other particles where it can react with other odorous substances and can decompose them into odor-neutral substances. This means a considerable improvement of the filtering effect therefore is caused by producing, or in general, by making available ozone in the outgoing airflow directly prior to entering the active carbon filtering material.

In addition, the reaction of ozone with other molecules retained in the active carbon filtering material releases reaction energy, which, depending on the reaction that occurs, can cause or at least facilitate a regeneration of the active carbon material. At the same time the active carbon filtering material is at least partially cleaned during the filtering process or the reduction of the filtering effect is at least delayed.

Advantageously, the airflow first flows through a particle filter before the particles contained therein are at least partially ionized and/or dissociated and the airflow then enters the odor filter. By arranging a particle filter first, large contamination pieces are prevented from penetrating the subsequent filter sections. Said large contamination pieces result in a premature saturation of the filtering material that cannot he regenerated or is tedious to regenerate, which could impede the filtering effect. This also avoids the absorption of an undesirably large share of energy supplied for the purpose of ionizing or dissociating odorous substances by particles, in particular dust particles or grease droplets, which would mean the energy is no longer available for filtering out odorous substances.

The method according to the invention not only can be used for filtering odorous substances out of an outgoing airflow but also for filtering an airflow in the circulating air or for filtering incoming air for rooms with special requirements concerning the purity of the ambient air.

The invention also relates to a filtering device for an airflow with an odor filter, said odor filter comprising active carbon or such as filtering material.

According to the invention, the filtering device has an energy-supplying device for the ionization and/or dissociation of the particles contained in the airflow, said energy-supplying device being arranged along the airflow in front of the odor filter. The filtering device can also comprise a UV radiation source that provides the energy.

In an especially advantageous manner the energy supplying device comprises two electrodes arranged at a distance from one another between which alternate voltage can be applied. By dimensioning and shaping the electrodes as well as triggering the alternate voltage applied between the electrodes, the ionization or dissociation of the particles that flow between the electrodes with the air can be set across a wide area and thus can adjust to the respective requirements or the odorous substances that are present in the airflow in especially high concentrations.

According to an advantageous embodiment of the inventive idea, the energy-supplying device comprises two electrodes arranged at a distance from one another between which a pulsating direct current or a direct current with superimposed alternating current can be applied. The electrodes preferably can substantially be in the form of a plane plate or plane grid. It also is possible to arrange a plurality of electrodes at a distance from each other in stacked fashion in the airflow.

Dielectric material to prevent discharge is arranged between the electrodes. It is common knowledge that a dielectrically impeded discharge provided by the arrangement of an dielectric or an insulator between the electrodes results in a markedly improved ionization and dissociation and also in a simpler and more effective generation and maintenance of a plasma. In the simplest case, one of two electrodes has an isolation layer.

Preferably, the filtering device comprises a particle filter that is arranged in the direction of flow in front of the energy-supplying device and the subsequent odor filter. Such a coarse particle filter, for example a grease or volume separator, provides an easy method for filtering out and separating a large part of the contaminants in the airflow. Depending on the intended use and the requirements on the filtering device that are known in advance, the particle filter can comprise a plurality of stages to achieve a gradual separation of fine and finest particles and contaminants in the airflow. The particle filter can also comprise additional sprinkler or washing equipment with a water mist or detergent.

In a particularly advantageous manner, the particle filter is at least partially open pore, hydrophilic cellular foam material. It was found that such filtering, materials have very good filtering properties for grease and steam droplets and that at the same time the filtered out contamination can efficiently be washed out and separated from the filtering material. If the airflow not only carries dry, dusty contamination but also has a sufficient share of steam and liquid droplets, the liquid amount that is absorbed in the open pore, hydrophilic foam material is sufficient to largely wash the biggest portion of all contaminants captured in the filtering material out of the filtering material so that the contaminants captured and accumulated in the open pore, hydrophilic foam material can be removed in a simple and easy manner.

It was found that filtering out large particles such as steam and grease droplets with a diameter of 1 micrometer or mote means that the subsequent energy-supplying device and odor filter are contaminated very little even over long periods of time and that their function is not compromised. Such a filtering device thus can be used in commercially and industrially used ventilation hoods without requiring disproportionately high maintenance. Such a combination of individual filtering elements not only allows the almost complete filtering out of large particles such as grease droplets or steam but also of very small particles and odors from the airflow that are difficult to filter out in a mechanical manner.

For the use in airflows, which carry a considerable share of dust, it may be practical to arrange a dust filter as a particle filter. A dust filter can be comprised of, for example, a fleece or layers of textile or metal materials.

In the following paragraphs a more detailed, schematic explanation of an exemplary embodiment for a filtering device according to the invention, which is described in the drawing, is given.

The FIGURE shows a schematic design of a filtering device arranged in an outgoing airflow channel 1. The filtering device first has a particle filter 2 in the direction of flow in which the outgoing airflow must flow through a layer of at least partially open pore, hydrophilic foam material. In this particle filter, 2 steam and grease droplets together with other particle shaped contaminants are separated from the airflow. However, most of the odorous substances cannot be captured and filtered out by such a mechanical particle filter.

Then the outgoing airflow is guided through an energy-supplying device 3 that comprises, in the direction of flow, a first plate electrode 4 and arranged at a distance from it a second plate electrode 5 so that the airflow can flow between both plate electrodes 4, 5. One or both plate electrodes 4, 5 can be part of the airflow channel 1 and do not necessarily need to cause a change of the cross section of the outgoing airflow channel 1. Alternating current is applied between the two plate electrodes 4, 5 with the help of a high-voltage transformer 6. The frequency of the alternating current is 50 Hz with other frequencies, in particular higher frequencies, being feasible and suitable for individual applications.

The airflow that is guided through the energy-supplying device 3 is at least partially ionized or dissociated inside.

Then the airflow is guided through an odor filter 7. The odor filter 7 contains filtering material 8 comprised of active carbon. The odor filter 7 not only captures and filters out particles, especially odorous substances, from the airflow that have not yet been filtered out but also captures and filters out the reaction products such as ozone produced by the energy-supplying device 3. The highly reactive reaction products that are generated and accumulated shortly before in the odor filter 7 facilitate additional reactions in the odor filter 7 that result in an additional reduction and filtering out of odorous substances.

The active carbon can be comprised of a plate module, a bulk filter or cartridge in the odor filter 7. Instead of the arrangement of two plate electrodes 4, 5 shown in the FIGURE, it is possible to arrange a plurality of electrodes in a stacked manner. The energy-supplying device 3 also can comprise a device for generating a plasma or a UV radiation source.

1-15. (canceled)

16. Method for filtering odors out of an airflow comprising
guiding an airflow containing odorous substances through a filtering device equipped with an odor filter having a substance serving as filtering material,
generating a plasma and at least partially dissociating at least a part of particles contained in the airflow upstream of the odor filter.

17. Method according to claim 1, wherein electric discharges are generated in the airflow before entering the odor filter.

18. Method according to claim 2, wherein the electric discharges are impeded by using a dielectric.

19. Method according to claim 1, wherein the airflow first is guided through a particle filter before an at least partial at least one of ionization and dissociation of the particles contained therein occurs and the airflow then enters the odor filter.

20. Filtering device for an airflow, the device comprising
an odor filter containing a substance serving as a filtering material, and
a plasma generating energy-supplying device adapted to dissociate particles contained in the airflow, the energy-supplying device being arranged along the airflow in front of the odor filter.

21. Filtering device according to claim 5, wherein the energy-supplying, device comprises two plate electrodes arranged at a distance from one another, between which an alternating current is applied.

22. Filtering device according to claim 5, wherein the energy-supplying device comprises two plate electrodes arranged at a distance from one another between which a pulsating direct current with superimposed alternating current can be applied.

23. Filtering device according to claim 7, wherein discharge impeding dielectric material is arranged between the electrodes.

24. Filtering device according to claim 5, wherein the filtering device comprises a particle filter that is arranged in the direction of flow in front of the energy-supplying device and the subsequent odor filter.

25. Filtering device according to claim 24, wherein the particle filter is an at least partially open pore, hydrophilic foam material.

26. Filtering device according to claim 24, wherein the particle filter is a dust filter.
